(19) United States
(12) Patent Application Publication
(10) Pub. No.: US 2026/0345852 A1
(43) Pub. Date: Nov. 5, 2026

(54) AROMA DIFFUSER

(71) Applicant: AROMA PERFECTO LLC, Diamond Bar, CA (US)

(72) Inventor: Yong WU, Guangzhou (CN)

(73) Assignee: AROMA PERFECTO LLC, Diamond Bar, CA (US)

(21) Appl. No.: 19/663,712

(22) Filed: Apr. 30, 2026

(30) Foreign Application Priority Data
Apr. 30, 2025 (CN) 202520864169.5

(51) Int. Cl. A61L 9/14

(52) U.S. Cl. CPC A61L 9/14

(57) ABSTRACT

An aroma diffuser includes: a main body; an atomization head in sealing connection with the main body through a sealing element; a solution container coupled to the atomization head, wherein a pressure chamber is formed between the solution container and the main body; and a high-pressure air generator, configured to supply pressurized air to the atomization head for atomization through the pressure chamber. A control panel is further included, which is formed with an opening for passing the atomization head and the solution container therethrough. The control panel includes a display layer and a metal support layer arranged from top to bottom.

### CROSS-REFERENCE TO RELATED APPLICATIONS

The present disclosure claims priority to Chinese Patent Application No. 202520864169.5, filed on Apr. 30, 2025, the content of which is incorporated herein by reference in its entirety.

### TECHNICAL FIELD

The present disclosure relates to the field of atomization devices, and more particularly, to an aroma diffuser.

### BACKGROUND

Aroma diffusers are widely used household appliances that convert aroma solution into fine mist for aromatherapy, humidification, and air purification. Among these, high-pressure air atomization diffusers are particularly popular due to their high efficiency and rapid aroma dispersion.

In the high-pressure air atomization diffusers, aroma liquid is suctioned, due to negative pressure caused by high-pressure air, into a mixing chamber, where the liquid and high-speed air are atomized into fine particles, and finally released as a mist to create a pleasant aromatic atmosphere.

In practical applications, particularly when the aroma diffusers feature a detachable structure, such as for replacing aroma solution containers, providing proper conduit alignment and sealing during high-pressure air delivery has become a pressing technical challenge. To address this issue, an approach is to provide a pressure chamber within the diffuser for containing the aroma solution container and the atomization assembly, which significantly simplifies the high-pressure air piping layout, thereby reducing production efforts and cost, as well as ensuring the overall structural compactness and aesthetic appearance of the aroma diffuser.

Although the aforementioned approach has achieved remarkable achievements in simplifying piping arrangements, it exposes significant deficiencies regarding the sealing between the atomization assembly and the pressure chamber. Such type of aroma diffusers usually achieves sealing through a threaded connection combined with a sealing ring. However, during the threading process, the atomization assembly exerts prolonged compression and friction on the sealing ring. This not only accelerates wear on the sealing ring, but also drastically shortens its service life, thereby compromising the overall performance and user experience of the aroma diffuser.

To overcome the sealing issues associated with threaded connections, some aroma diffusers achieve sealing through a snap-fit structure with a sealing ring. However, snap-fit structures, such as snap lugs and snap grooves, usually require an interference fit to effectively abut against the sealing element for a tight sealing. Since the atomization head and the main body are typically made of plastic materials, they are prone to deformation under the force applied thereon. To mitigate the reduction in sealing performance caused by such deformation, the interference amount may be further increased, resulting in that greater force is required to overcome the interference fit during installation and disassembly of the atomization head and the main body, which adversely affects the user experience.

Additionally, such aroma diffusers typically arrange the touch control panel surrounding the atomization head. This design not only presents a visually concise and aesthetic appearance, but also allows for a rational layout of the operation area within limited space, facilitating operation of the control panel. To ensure a good tactile feel and stable electrical connection, the touch control panel is required to rest against the plastic support on the main body.

However, in actual production and manufacturing, due to various factors, it is difficult to achieve an ideal surface flatness for the plastic support. As a result, during the process of attaching the flexible control circuit onto the plastic support, defects such as warping or air bubbles are highly likely to occur. Warping can lead to local gaps between the flexible control circuit and the plastic support, causing unstable signal transmission during touch operations. This may result in issues like false touches or unresponsiveness, severely affecting the use experience. The formation of air bubbles on one hand impacts the aesthetic appearance of the aroma diffuser, on the other hand further interferes with the electrical connection of the flexible control circuit, reducing the sensitivity and reliability of the touch control panel.

### SUMMARY

The present disclosure therefore provides an improved aroma diffuser, which has enhanced structural rigidity, sealing performance, and user experience.

The diffuser according to the present disclosure includes a main body, an atomization head, and a solution container for containing a solution to be atomized. The atomization head is coupled to the solution container, and configured to draw the solution from the solution container and mix it with pressurized air to form a mist, which is finally released from the atomization head. A sealing element is further arranged between the atomization head and the main body to achieve a sealed connection therebetween. A pressure chamber is formed between the solution container and the main body. A high-pressure air generator is further included to supply the pressurized air to the pressure chamber, and the pressurized air is further delivered to the atomization head. A control panel is arranged on the main body. An opening is formed in the control panel for passing the atomization head and the solution container therethrough. A control assembly is further provided in the main body, which is in control connection with the control panel. In addition, the control panel comprises a display layer and a metal support layer arranged from top to bottom.

The annular control panel with an opening is usually prone to deformation during installation, resulting in an uneven surface of the control panel. In the diffuser of the present disclosure, the control panel includes a display layer and a metal support layer arranged from top to bottom. The metal support layer can prevent the display layer from deforming, making the entire surface of the control panel flat and aesthetic after installation. As mentioned above, for the case of the atomization head and the main body, which are made of plastic materials, being in sealing connection through a sealing element, the interference amount is usually significantly increased to alleviate the sealing performance deterioration caused by deforming, which in turn requires more efforts to overcome the interference fit during assembly and disassembly. However, the metal support layer provided in the present disclosure can effectively prevent the plastic parts from deforming due to higher deformation resistance, the desired sealing effect of the diffusion thus can be achieved without remarkably increasing the interference amount, thereby providing better use experience during disassembly and assembly.

To further improve the sealing performance, the atomization head and the main body may be coupled by 2 to 5 circumferentially distributed snap-fit elements, especially uniformly distributed. Each snap-fit element may particularly include a snap lug protruding from the atomization head and a snap groove recessed in the main body. The control panel may further include a holder arranged below the metal support layer, and the snap groove is formed in the holder, especially at the inner side of the holder.

At least three snap-fit elements may form a stable fastening structure, while at most five snap-fit elements may not unduly increase the complexity in structure and facilitate fastening. In assembly, the atomization head is moved down to press against the sealing element together with the main body, the snap lug of the atomization head is snapped into the snap groove of the main body, achieving stable connection between the atomization head and the main body. In a particular embodiment, the snap lug of the atomization head may be first vertically inserted into a vertical section of the snap groove, and then transversely inserted into a transverse section of the snap groove by rotating the atomization head. The transverse section can provide improved fastening support, thereby achieving a stable connection between the atomization head and the main body, as well as ensuring the sealing of the pressure chamber. Since the snap-fit element do not exert excessive extrusion and friction on the sealing element during the fitting process, the service life of the sealing element can be effectively prolonged. In addition, the snap groove is formed in the holder, and the holder is isolated from the display layer by the metal support layer, which can also effectively ensure that the deformation of the holder under stress will not be transmitted to the display layer, maintaining the display layer flat during use.

In a preferable embodiment of the present disclosure, the metal support layer may be fixed to the holder by fasteners, and the fasteners may be arranged circumferentially around the opening and corresponding to the snap-fit elements. In this configuration, the fasteners also serve to enhance the fitting stability between the respective snap lug and the snap groove. By arranging the fasteners corresponding to the snap-fit elements, one or more snap grooves can be prevented from deforming upward under the reaction force during the fitting process, ensuring that the atomization head and the main body can form a stable pressing plane for the sealing element even that multiple disassembly and assembly have been performed. The fasteners may be generally screws matched with screw holes in the holder, and the screw holes may be circumferential positioned at the outer side of the positions corresponding to the snap-fit elements.

In a particular embodiment, the snap groove may be generally L-shaped and particularly include a vertical section allowing the snap lug to be vertically inserted and a transverse section allowing the snap lug to be transversely inserted, where when the snap lug is snapped into the snap groove, the atomization head and the main body are pressed against the sealing element to achieve sealing. In the context of the present disclosure, in order to increase pressing against the sealing element, the transverse section of the snap groove may be also slightly inclined downward at a certain angle, or inclined downward at a certain angle and further transversely extended. This exhibits higher stability of the snap fitting.

Furthermore, a transitional section having an inclined or arcuate surface may be further formed between the vertical section and the transverse section. The snap lug may also be correspondingly chamfered. This provides a smoother transition during the rotation of the snap lug from the vertical section to the transverse section, thereby reducing friction resistance between the snap lug and the snap groove, and thus making the fitting process smoother. In addition, the distance between the atomization head and the main body may be further reduced, allowing the sealing element to be further pressed, thereby further improving the sealing performance.

The main body may particularly include an inner cavity and a housing. In this case, the inner cavity may be arranged in the housing, and the solution container may be positioned in the inner cavity. The sealing element may be arranged at the upper rim of the inner cavity to seal against the atomization head. The pressure chamber may be formed between the solution container and the inner cavity. The inner cavity is connected to the holder, especially the lower side thereof, and the two can be fixed by the above fasteners. The holder and the upper edge of the inner cavity may jointly fix the sealing element. The high-pressure air generator may be arranged between the inner cavity and the housing, which is communicated with the pressure inner cavity through an air supply pipe. This makes the entire equipment more compact in structure. In addition, this also facilitates integration of the internal components, such as the holder, inner cavity, and high-pressure air generator, with the control panel, the integrated component thus can be integrally embedded into the housing.

The above integrated component can be embedded into the housing in two different ways.

In a first way, the upper portion of the housing may be formed with a support shoulder, and the holder may be supported on the support shoulder. In this case, the inner cavity is supported inside the outer housing through the holder. In this way, the components such as the inner cavity and the high-pressure air generator may be suspended at the upper portion of the housing through the holder, the holder may support the control panel on one side and support the atomization head and solution container on the other side. This requires high strength for the holder.

In a second way, the upper portion of the housing may be formed with a support shoulder, the upper portion of the inner cavity may be radially extended toward the housing to form a support, the support may be formed with a flange, and the flange may be supported on the support shoulder. In this case, the inner cavity is supported inside the housing through the flange. In this way, the support extended from the inner cavity is supported on the housing, the holder and the control panel are supported on the support, and the atomization head and the solution container are supported by the holder. This requires lower strength of the holder.

In a preferable embodiment of the present disclosure, a buffer chamber may be further provided, where the pressurized air may be supplied from the high-pressure air generator into the pressure chamber through the buffer chamber. The buffer chamber can reduce the flow rate of the pressurized air, making its pressure distribution more uniform, thereby improving pressure stability in the pressure inner cavity. This can further effectively absorb and disperse the noise generated by pressurized air thereby reducing the operation noise of the equipment. The aroma liquid can also be prevented from entering the high-pressure air generator, thereby improving the use experience.

In a particular embodiment of the present disclosure, the buffer chamber may be formed surrounding the inner cavity, with one air inlet connected to the air supply pipe and multiple air outlets connected to the inner cavity. The annular buffer chamber can provide improved structural support and enhance overall stability. The arrangement of multiple air outlets enables high-pressure air to be more uniformly distributed in the inner cavity.

The multiple air outlets may be preferably slit-shaped, extending vertically downward from the upper portion of the inner cavity, with the lower ends of the air outlets maintaining a certain distance from the underside of the buffer chamber. On one hand, the slit-shaped air outlets may ensure the required size of the air outlets to avoid excessively high or low local pressure. On the other hand, as excessively large openings usually cause strength reduction of the side wall of the buffer chamber, the slit-shaped air outlets thus may also ensure good structural performance. In addition, the slit-shaped air outlets maintain a certain distance from the bottom of the buffer chamber, forming a cavity at the bottom of the buffer chamber. Accordingly, when occasionally entering the buffer chamber, the atomized aroma liquid can be condensed in the buffer chamber and may not flow into the air supply pipe. This ensures normal operation of the diffuser.

More preferably, the inner cavity, the support, and the buffer chamber may be integrally formed. The integral structure, especially formed by injection molding, may not only reduce the number of components and assembly effort, thereby improving production efficiency, but also ensures the overall strength of the support.

According to the present disclosure, the configuration of the metal support layer may improve the use experience and service life of the aroma diffuser, as well as the production yield. The improved sealing element may significantly enhance the sealing performance of the inner cavity. The connection portion of the sealing element may serve as a reference for installation and positioning, thereby simplifying the assembly process. Additionally, the configuration of the compression portion may enable the sealing element to be pressed and fixed by other internal structures of the main body while being installed on the upper rim of the inner cavity, thereby preventing the sealing element from shifting during repeated compression. The metal support layer of the panel can prevent the display layer of the panel from deforming, making the entire control panel flat and aesthetic after installation. In addition, the design of the holder not only supports the control panel, but also jointly press against the compression portion of the sealing element with the inner cavity, further improving the structural stability and sealing performance.

The above and other features of the present disclosure, including various novel details of construction and combinations of parts, will now be more particularly described with reference to the accompanying drawings and pointed out in the claims. It will be understood that the particular methods and apparatus embodying the disclosure are shown by way of illustration and not as a limitation of the disclosure.

### BRIEF DESCRIPTION OF THE DRAWINGS

FIG. 1 is a perspective view of an aroma diffuser according to an embodiment of the present disclosure.

FIG. 2 is an exploded view of the aroma diffuser in FIG. 1.

FIG. 3 is a front view of the aroma diffuser in FIG. 1.

FIG. 4 is a cross-sectional view of the aroma diffuser taken along line A–A of FIG. 3.

FIG. 5 is a partial assembly view of the aroma diffuser in FIG. 1, with some parts omitted.

FIG. 6 is an enlarged detail showing the assembly of the main body 100 and an atomization head 200, with some parts omitted.

FIG. 7 is a perspective view of a main body of the aroma diffuser in FIG. 1.

FIG. 8 is an enlarged detail view of portion I in FIG. 7.

FIG. 9 is a perspective section view of the main body of the aroma diffuser along line A–A in FIG. 3.

FIG. 10 is an enlarged detail view of portion II in FIG. 9.

FIG. 11 is another perspective view of the main body of the aroma diffuser in FIG. 1.

FIG. 12 is an enlarged detail view of portion III in FIG. 11.

FIG. 13 is a structural view showing a buffer chamber.

FIG. 14 is a perspective section view of a sealing element according to an embodiment of the present disclosure.

FIG. 15 is a perspective section view of the aroma diffuser taken along line A–A of FIG. 3, with some parts omitted.

FIG. 16 is an enlarged detail view of portion IV in FIG. 15.

FIG. 17 is another perspective view of the main body of the aroma diffuser in FIG. 1.

FIG. 18 is a cross‑sectional view of an aroma diffuser according to another embodiment of the present disclosure.

FIG. 19 is a perspective section view of the main body in FIG. 18.

FIG. 20 is an enlarged detail view of portion V in FIG. 19.

FIG. 21 is a perspective section view of a sealing element of according to another embodiment of the present disclosure.

FIG. 22 is a top view of the aroma diffuser in FIG. 18.

FIG. 23 is a perspective section view taken along line B–B of FIG. 22.

FIG. 24 is an enlarged detail view of portion VI in FIG. 23.

### DETAILED DESCRIPTION OF THE EMBODIMENTS

The drawings are merely for illustrative purposes and should not be interpreted as limitations to this patent. To better illustrate the embodiments, some components in the drawings may be omitted, enlarged, or reduced, and do not represent the actual size of the product. For those skilled in the art, it can be understood that some well-known structures and their descriptions may be omitted in the drawings. The positional relationships depicted in the drawings are for illustrative purposes only and are not to be construed as limiting this patent.

Reference will now be made in detail to embodiments of the present disclosure, examples of which are illustrated in the accompanying drawings

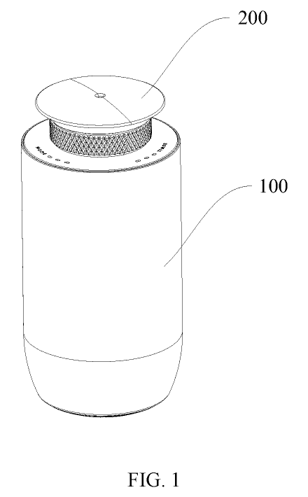

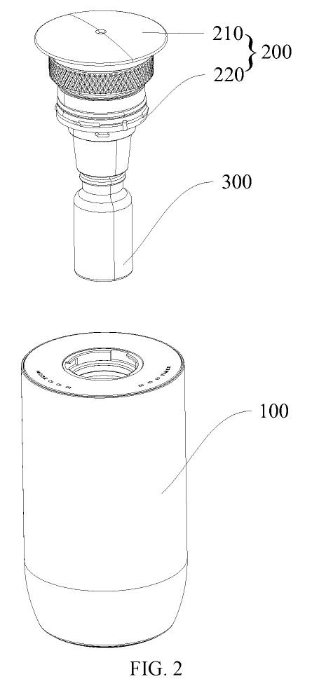

FIG. 1 provides an aroma diffuser according to an embodiment of the present disclosure, including a main body 100 and an atomization head 200. Referring to FIG. 2, the atomization head 200 particularly includes a cover 210 and an atomization assembly 220. The atomization assembly 220 is coupled to a solution container 300 for containing the solution to be atomized. The atomization assembly 220 is configured to draw the solution from the solution container 300 and mix it with the pressurized air to form a mist, which is finally released from the cover 210.

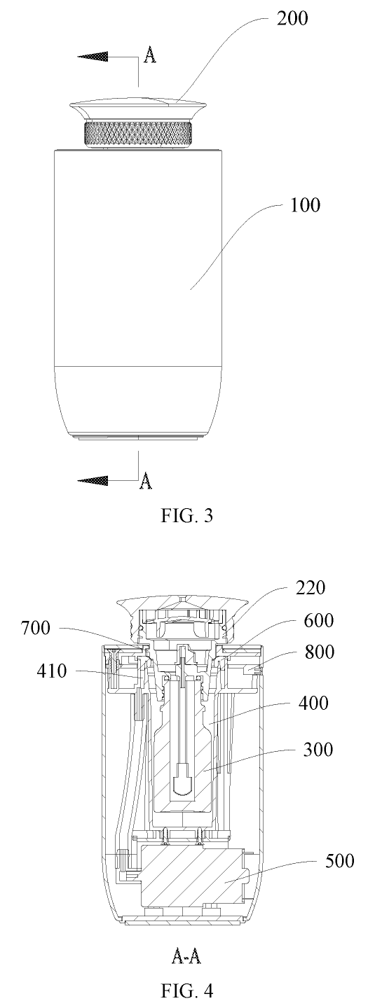

In combination with FIGS. 3, and 4, the atomization head 200 is in sealing connection with the main body 100, especially through a sealing element 600. A pressure chamber 400 is formed between the solution container 300 and the main body 100. A high‑pressure air generator 500 for supplying pressurized air to the pressure chamber 400 is further arranged at the bottom of the main body 100. The pressurized air is finally fed to the atomization assembly 220 from the pressure chamber 400. A control module 800 is further provided within the main body 100.

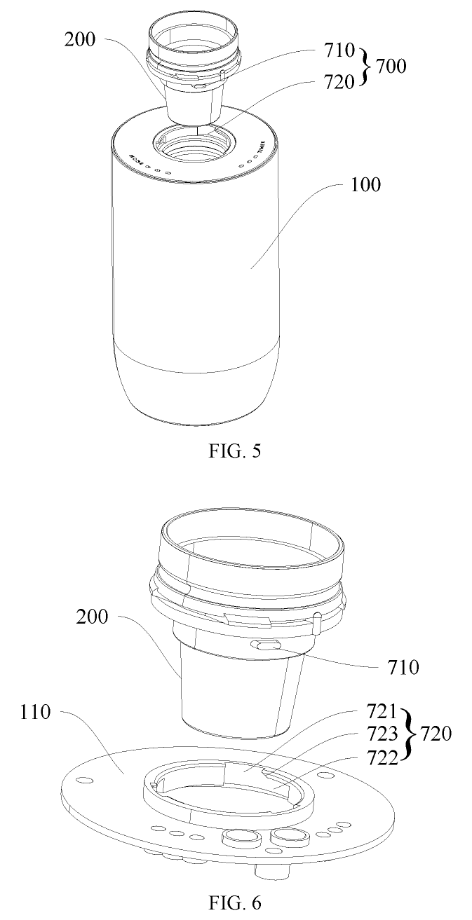

In combination with FIG. 2 and FIG. 5, The atomization head 200 is coupled to the main body 100 by three circumferentially uniformly distributed snap‑fit elements 700 in this embodiment. Each snap‑fit element 700 includes a snap lug 710 protruding from the atomization head 200, and a snap groove 720, which substantially has an L-shaped cross section, recessed in the main body 100. Referring to FIG. 6, the snap groove 720 particularly includes a vertical section 721 allowing the snap lug 710 to be vertically inserted, and a transverse section 722 allowing the snap lug 710 to be transversely inserted, and a transitional section 723 connecting the vertical section 721 to the transverse section 722. The transitional section 723 in this embodiment has an inclined surface. In such configuration, when the snap lug 710 is snapped into the snap groove 720, the atomization head 200 and the main body 100 are pressed against the sealing element 600 to achieve sealing.

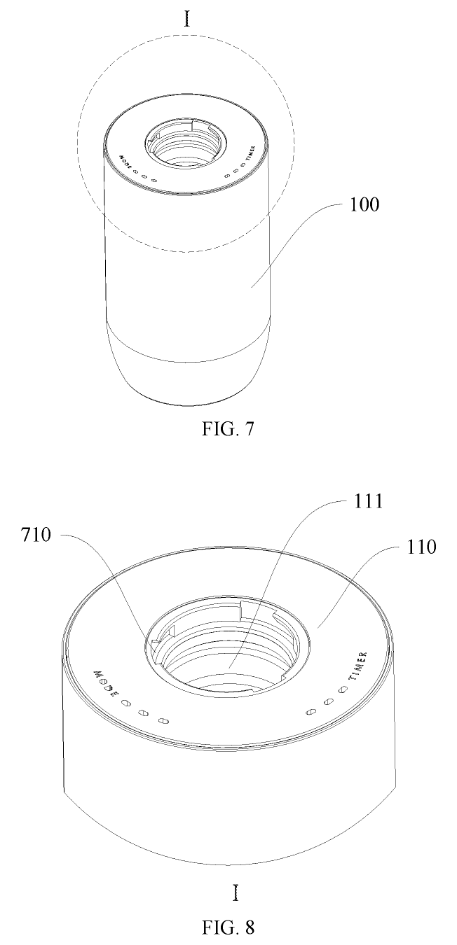

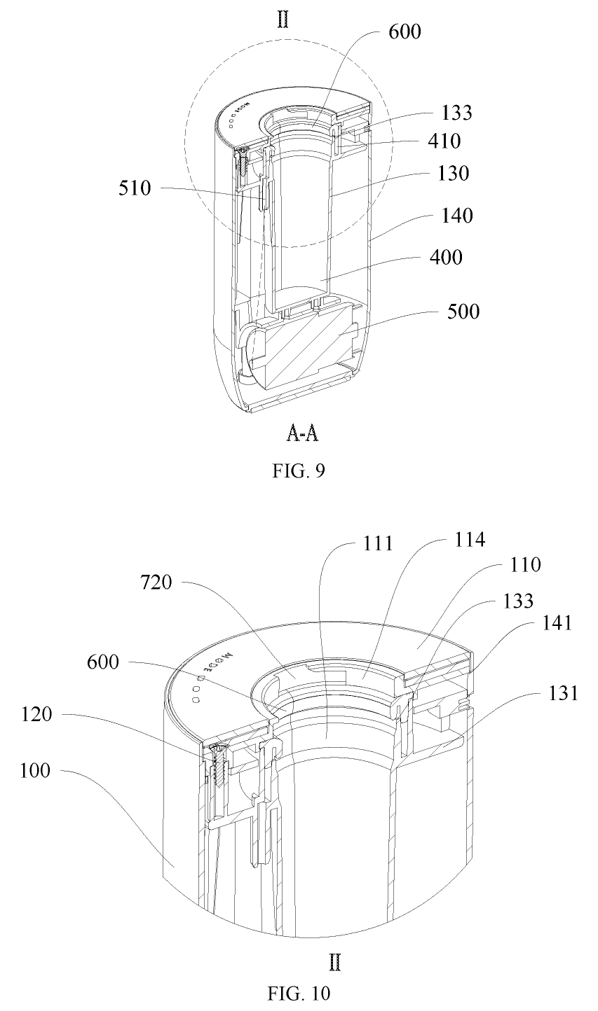

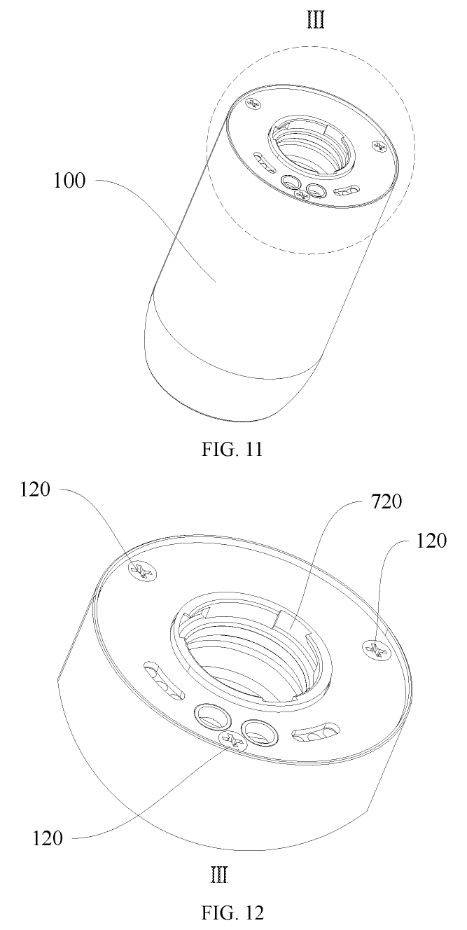

In combination with FIGS. 7, and 8, a control panel 110 is provided on the upper end of the main body 100. The control panel 110 is formed with a central opening 111 for the atomization head 200 and the solution container 300 passing therethrough. The snap grooves 720 are circumferentially formed on the inner wall of the opening 111. Further in combination with FIGS. 9, and 10, the control panel 110 is fastened to the main body 100, especially by fasteners 120. Further in combination with FIGS. 11, and 12, the fasteners 120 are arranged circumferentially around the opening 111 and corresponding to the snap‑fit elements 700.

Referring back to FIG. 9, the main body 100 in this embodiment particularly includes an inner cavity 130 and a housing 140 for containing the inner cavity 130. As shown in FIG. 4, the solution container 300 is positioned inside the inner cavity 130. The sealing element 600 is positioned at the upper rim 133 of the inner cavity 130 to seal against the atomization assembly 220. The pressure chamber 400 is particularly formed between the solution container 300 and inner cavity 130. The high‑pressure air generator 500 is disposed between the inner cavity 130 and the housing 140, which is communicated with the pressure chamber 400 via an air supply pipe 510. A buffer chamber 410 is further provided. In such configuration, the pressurized air from the high‑pressure air generator 500 is directed into the pressure chamber through the buffer chamber via the air supply pipe 510.

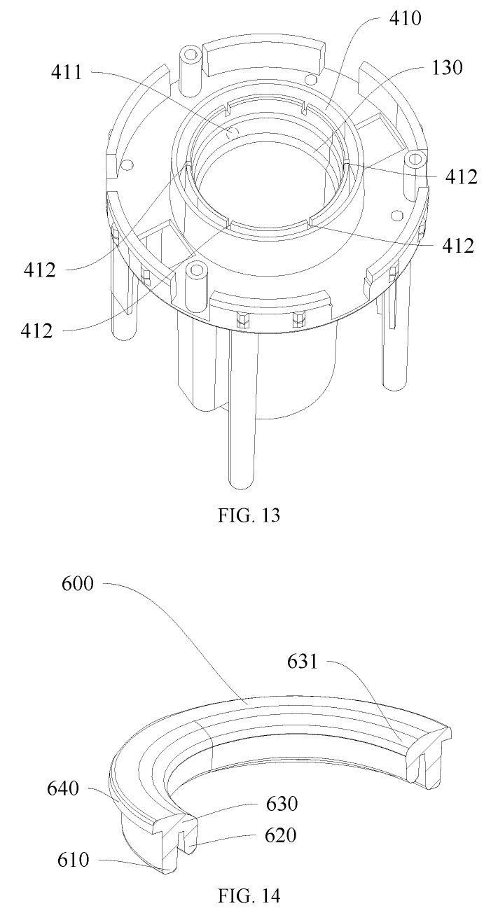

Referring now to FIG. 13, the buffer chamber 410 is formed surrounding the inner cavity 130, with an inlet 411 communicated with the air supply pipe 510 and a plurality of outlets 412 communicated with the inner cavity 130. In this embodiment, the outlets 412 are slit‑shaped, which are vertically extended down from the upper end of the inner cavity 130, especially from the upper rim of the inner cavity 130, with a certain distance between the lower end of the outlets 412 and the underside of the buffer chamber 410, namely the lower end of the outlets 412 is not flush with the underside of the buffer chamber 410.

In combination with FIGS. 10, and 14, the sealing element 600 in this embodiment particularly includes an outer sealing ring 610 engaged around the outer side of the upper rim 133 of the inner cavity 130, an inner sealing ring 620 engaged around the inner side of the upper rim 133 of the inner cavity 130, and a connection portion 630 integrally connecting the upper end of the outer sealing ring 610 to the upper end of the inner sealing ring 620. The outer sealing ring 610 projects beyond the bottom end of the inner sealing ring 620. The connection portion 630 is formed with a transitional curved surface 631 extending toward the inner sealing ring 620. Furthermore, the connection portion 630 is radially extended outward from the outer sealing ring 610 to form a compression portion 640.

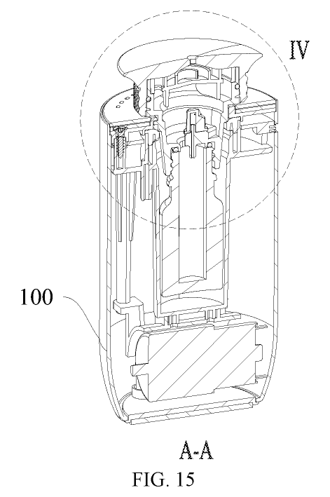

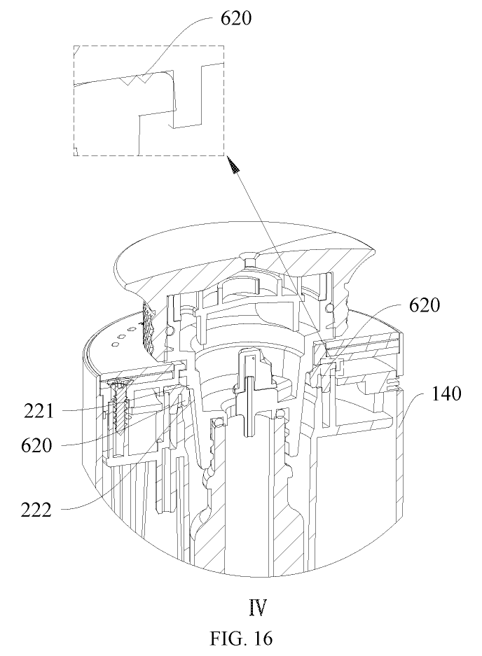

In combination with FIGS. 15, and 16, the compression portion 640 is pressed and fastened by the main body 100. A friction surface 641 is provided on the contact surface between the compression portion 640 and the main body 100 - especially on the compression portion 640 - to prevent relative movement therebetween. The atomization assembly 220 is formed with a pressing shoulder 221 corresponding to the inner sealing ring 620. The pressing shoulder 221 is pressed against the inner sealing ring 620.

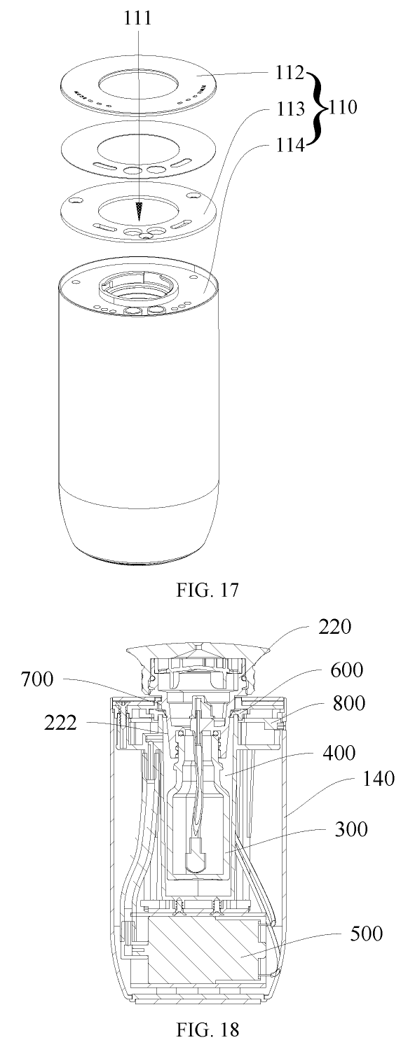

Referring to FIG. 17, the control panel 110 in this embodiment particularly includes a display layer 112, a metal support layer 113, and a holder 114 arranged from top to bottom. In conjunction with FIG. 10, the snap grooves 720 of the snap-fit elements 700 are formed at the inner side of the holder 114. Particularly, the holder 114 may be formed with a flange around the opening 111, which is perpendicular to the display layer 112 and the metal support layer 113. The snap grooves 720 may be preferably formed in the flange. The upper portion of the inner cavity 130 is extended toward the housing 140 to form a support 131. An upper portion of the housing 140 is formed with a support shoulder 141. The holder 114 is rested on the inner cavity 130 and the support shoulder 141. The sealing element 600 is clamped between the holder 114 and the inner cavity 130. In this embodiment, the inner cavity 130 is connected to the holder 114, especially the lower side of the holder 114. Accordingly, the inner cavity 130 in this embodiment is supported inside the housing through the holder 114.

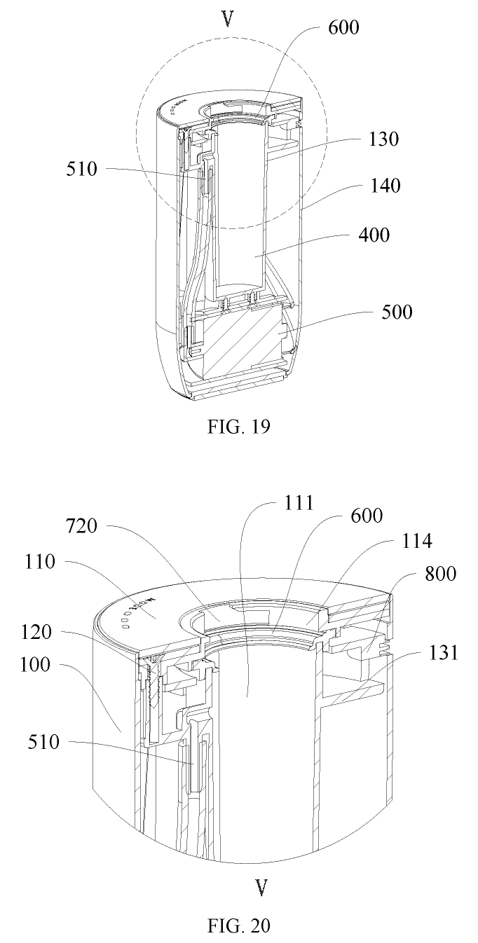

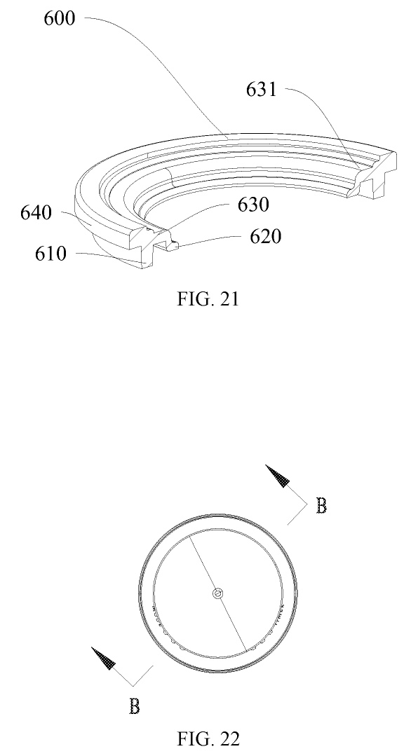

FIG. 18 shows a cross section of an aroma diffuser according to another embodiment of the present disclosure. FIG. 19 shows a cross section of the main body of the aroma diffuser in FIG. 18. In this embodiment, as clearly shown in FIG. 21, the buffer chamber 410 is not provided. That is, the air supply pipe 510 in this embodiment is directedly communicated with the pressure chamber 400 through the outlet 412.

Referring to FIG. 21, the inner sealing ring 620 of the sealing element 600 in this embodiment is further inwardly formed with a stepped portion. The lower portion of the atomization assembly 220 in this embodiment is designed as a tapered structure 222 (clearly shown in FIG. 18), which is abutted against the inner sealing ring 620, especially the stepped portion of the inner sealing ring 620.

In the present embodiment, the inner sealing ring 620 has an inner diameter smaller than the diameter of the opening 111. When the atomization assembly 220 is inserted into the opening 111 to engage with the main body 100, the tapered surface 222 progressively contacts the inner sealing ring 620 from a smaller diameter end to a larger diameter end. Upon contact, the tapered surface 222 presses the inner sealing ring 620 downwards, causing the stepped portion to deflect downwards. Due to good elastic restoring properties, the stepped portion is abutted tightly against the tapered surface 222 to achieve sealing between the atomization assembly 220 and the inner cavity 130.

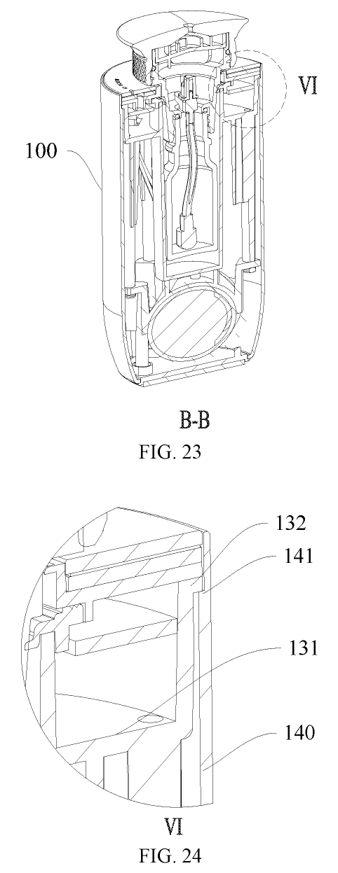

Referring to FIGS. 22 to 24, in this embodiment, the housing 140 is formed with a support shoulder 141 at the upper portion. Correspondingly, the support 131 of the inner cavity 130 is formed with a flange 132. The flange 132 is supported on the support shoulder 141 of the housing 140. In this embodiment, the holder 114 is supported on the support shoulder 141 of the housing 140. Accordingly, the inner cavity 130 in this embodiment is supported inside the housing through the support 131.

Apparently, the above embodiments of the present disclosure are only examples provided to clearly illustrate the present disclosure, and are not limitations on the implementations of the present disclosure. For those of ordinary skill in the art, various other forms of modifications or changes can be made on the basis of the above description. It is neither necessary nor possible to enumerate all embodiments herein. Any modifications, equivalent replacements, improvements, etc. made within the spirit and principle of the present disclosure shall be included within the scope of protection of the claims of the present disclosure.

1. An aroma diffuser, comprising:
a main body;
an atomization head, configured to form a sealed connection with the main body via a sealing element;
a solution container for containing a solution to be atomized, the solution container being coupled to the atomization head, wherein a pressure chamber is formed between the solution container and the main body; and
a high-pressure air generator, configured to supply pressurized air to the atomization head for atomization through the pressure chamber,
wherein the atomization head is configured to draw the solution from the solution container and mix the solution with the pressurized air to form a mist, and the mist is released from the atomization head,
the main body is provided with a control panel, the control panel is formed with an opening for passing the atomization head and the solution container therethrough, and the main body is further provided with a control module operatively coupled to the control panel, and
the control panel comprises a display layer and a metal support layer arranged from top to bottom.

2. The aroma diffuser according to claim 1, wherein the atomization head is coupled to the main body by 2 to 5 circumferentially distributed snap-fit elements, each of the snap-fit elements comprises a snap lug protruding from the atomization head and a snap groove recessed in the main body; and
the control panel further comprises a holder disposed below the metal support layer, and the snap groove is formed in an inner side of the holder close to the opening.

3. The aroma diffuser according to claim 2, wherein the metal support layer is secured to the holder by fasteners, and the fasteners are arranged circumferentially around the opening and corresponding to the snap-fit elements.

4. The aroma diffuser according to claim 2, wherein the snap groove is generally L-shaped and comprises a vertical section configured to allow the snap lug to be vertically inserted and a transverse section configured to allow the snap lug to be transversely inserted, and when the snap lug is snapped into the snap groove, the atomization head and the main body are pressed against the sealing element to achieve sealing.

5. The aroma diffuser according to claim 4, wherein a transitional section having an inclined or arcuate surface is formed between the vertical section and the transverse section.

6. The aroma diffuser according to claim 2, wherein the main body comprises an inner cavity for containing the solution container, and a housing for containing the inner cavity, and
the sealing element is disposed at an upper rim of the inner cavity to seal against the atomization head, the pressure chamber is formed between the solution container and the inner cavity, the inner cavity is connected to the holder, and the sealing element is secured between the holder and the inner cavity.

7. The aroma diffuser according to claim 6, wherein an upper portion of the housing is formed with a support shoulder, and the holder is supported on the support shoulder, such that the inner cavity is supported inside the outer housing through the holder.

8. The aroma diffuser according to claim 6, wherein an upper portion of the housing is formed with a support shoulder, an upper portion of the inner cavity is radially extended toward the housing to form a support formed with a flange, and the flange is supported on the support shoulder, such that the inner cavity is supported inside the housing through the support.

9. The aroma diffuser according to claim 6, wherein an upper portion of the inner cavity is radially extended toward the housing to form a support, a buffer chamber surrounding the inner cavity is formed on the support, and the pressurized air is supplied from the high-pressure air generator into the pressure chamber through the buffer chamber.

10. The aroma diffuser according to claim 9, wherein the inner cavity, the support, and the buffer chamber are integrally formed.
